(19) United States
(12) Patent Application Publication
(10) Pub. No.: US 2026/0188526 A1
(43) Pub. Date: Jul. 2, 2026

(54) DATA PROCESSING APPARATUS AND METHOD

(71) Applicant: CANON MEDICAL SYSTEMS CORPORATION, Otawara-shi (JP)

(72) Inventors: Owen ANDERSON, Edinburgh (GB); Russell HUNG, Edinburgh (GB); James LESH, Edinburgh (GB); Simon FISHER, Edinburgh (GB); Ian POOLE, Edinburgh (GB)

(73) Assignee: CANON MEDICAL SYSTEMS CORPORATION, Otawara-shi (JP)

(21) Appl. No.: 19/004,637

(22) Filed: Dec. 30, 2024

(51) Int. Cl. G16H 70/60; G16H 10/60

(52) U.S. Cl. CPC G16H 70/60; G16H 10/60

(57) ABSTRACT

A medical data processing apparatus comprises processing circuitry configured to:
receive a first medical text and extract at least one item from the first medical text;
receive a second medical text and determine whether or not there is a match between the extracted at least one item and content of the second medical text including if there is a match determining a part of the second text that matches the extracted at least one item.

### FIELD

Embodiments described herein relate generally to a method and apparatus for processing text, for example for training and using a model to match text from two or more sources.

### BACKGROUND

A number of LLMs including Generative Pre-trained Transformers (GPT) and Bard have entered into public use, with implications which are highly disruptive for many industries. Many of these models are available for use via API access, and several others are available for download to be run locally.

These models are trained on a corpus of text, generally obtained from the internet, in an unsupervised fashion, and are capable of solving complex linguistically expressed tasks such as note summarisation, answering exam questions, and writing essays. They are capable of consuming both structured and unstructured text. They may provide output structured in several formats, for example in the “.json” format.

Current LLMs are already broad in terms of their capabilities and will continue to improve. It is likely that in the future, such LLMs may be used to link together disparate modalities of data and reconcile them for the user through the intermediate format of language.

There are a number of key challenges which must be overcome for LLMs to be implemented in Precision Clinical Decision Support (P-CDS). The knowledge LLMs possess internally is likely to always be out of date, especially with respect to rapidly changing local health care guidelines, up to date medical publications and clinical knowledge. With regards to any clinical deployment, it is critical that LLMs can be constrained at deployment time to a specific and curated source of guidance and knowledge, which is up to date.

The workings of these models are often opaque to the user, which can be a significant drawback particularly in clinical settings.

LLMs are capable of hallucinating responses which sound highly plausible, in a manner which is difficult for a user to detect. It is important that the risk of such output reaching the user is mitigated, particularly in clinical applications.

### BRIEF DESCRIPTION OF THE DRAWINGS

Embodiments are now described, by way of non-limiting example, and are illustrated in the following figures, in which:

FIG. 1 is a schematic illustration of an apparatus in accordance with an embodiment;

FIG. 2 is a schematic of a method of processing text data in accordance with an embodiment;

FIG. 3 is a representation of a graphical user interface in accordance with an embodiment;

FIG. 4 is a schematic of a method of processing text data and an image of the associated graphical user interface in accordance with an embodiment;

FIG. 5 is a representation of a graphical user interface in accordance with an embodiment;

FIG. 6 is a schematic illustrating a method in accordance with an embodiment;

FIG. 7 is a representation of a graphical user interface in accordance with an embodiment;

FIG. 8 is a representation of a graphical user interface in accordance with an embodiment; and

FIG. 9 is a representation of a graphical user interface in accordance with an embodiment.

### DETAILED DESCRIPTION

According to certain embodiments there is provided medical data processing apparatus comprising processing circuitry configured to:
receive a first medical text and extract at least one item from the first medical text;
receive a second medical text and determine whether or not there is a match between the extracted at least one item and the content of the second medical text including if there is a match determining a part of the second text that matches the extracted at least one item.

According to certain embodiments there is provided a method of matching medical data texts comprising:
receiving a first medical text and extract at least one item from the first medical text;
receiving a second medical text and determine whether or not there is a match between the extracted at least one item and content of the second medical text including if there is a match determining a part of the second text that matches the extracted at least one item.

According to certain embodiments there is provided a non-transitory computer program product storing computer-readable instructions that are executable to:
receive a first medical text and extract at least one item from the first medical text;
receive a second medical text and determine whether or not there is a match between the extracted at least one item and content of the second medical text including if there is a match determining a part of the second text that matches the extracted at least one item.

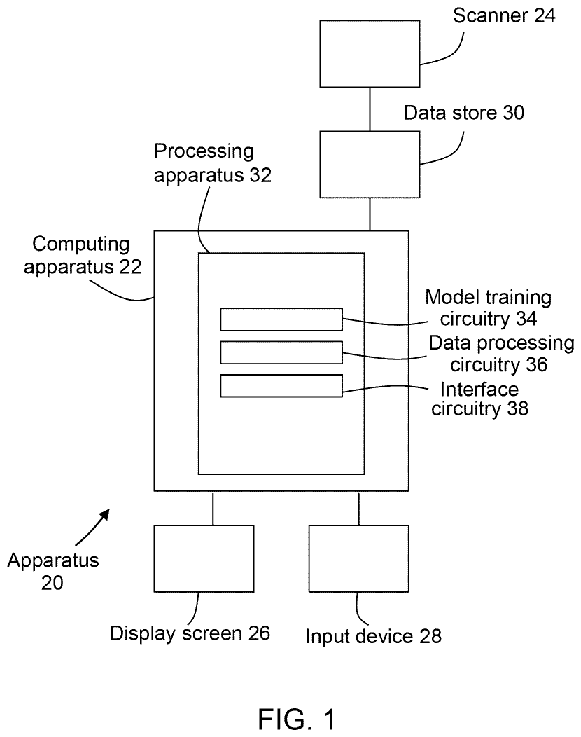

A data processing apparatus 20 according to an embodiment is illustrated schematically in FIG. 1. In the present embodiment, the data processing apparatus 20 is configured to process text data. In other embodiments, the data processing apparatus 20 may be configured to process any other appropriate data.

The data processing apparatus 20 comprises a computing apparatus 22, which in this case is a personal computer (PC) or workstation. The computing apparatus 22 is connected to a display screen 26 or other display device, and an input device or devices 28, such as a computer keyboard and mouse.

The computing apparatus 22 is configured to obtain data sets from a data store 30. The data sets have been obtained or generated using any suitable apparatus or from any suitable source. In some embodiments, at least some of the data can include, or can be determined from medical report data, for instance obtained using a scanner 24.

The computing apparatus 22 may receive data from one or more further data stores (not shown) instead of or in addition to data store 30. For example, the computing apparatus 22 may receive medical image data from one or more remote data stores (not shown) or other information system. Computing apparatus 22 provides a processing resource for automatically or semi-automatically processing the data. Computing apparatus 22 comprises processing circuitry 32. The processing circuitry 32 comprises application program interface (API) and communication circuitry 34, data processing circuitry 36 configured to perform processes including providing data to and receiving data from the API circuitry as part of such processes, and interface circuitry 38 configured to obtain user or other inputs and/or to output results of the data processing via a user interface.

In the present embodiment, the circuitries 34, 36, 38 are each implemented in computing apparatus 22 by means of a computer program having computer-readable instructions that are executable to perform the method of the embodiment. However, in other embodiments, the various circuitries may be implemented as one or more ASICs (application specific integrated circuits) or FPGAs (field programmable gate arrays).

The computing apparatus 22 also includes a hard drive and other components of a PC including RAM, ROM, a data bus, an operating system including various device drivers, and hardware devices including a graphics card. Such components are not shown in FIG. 1 for clarity. The data processing apparatus 20 of FIG. 1 is configured to perform methods as illustrated and described in the following.

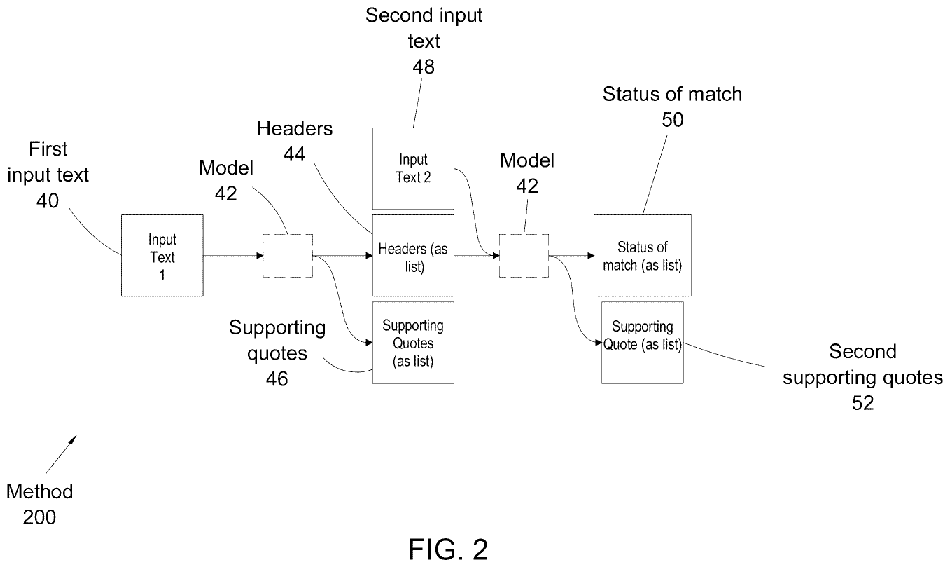

FIG. 2 is a schematic of a method 200 of processing text data in accordance with an embodiment, performed under control of the processing circuitry 32 of FIG. 1. With reference to FIG. 2, a first input text 40 is provided to a model 42. The first input text 40 may be structured text or unstructured text or a combination of structured and unstructured text. Unlike structured text data which comprise an inherent structure that makes extraction of relevant information easier, free text is unstructured and commonly requires one or more of higher processing power and a larger amount of context to process text. Electronic healthcare records contain large volumes of unstructured data in different forms. Free text constitutes a large portion of such data.

The model 42 that processes the first input text 40 is a trained machine learning model. The model 42 comprises a large language model (LLM) in the embodiment of FIG. 2. The model is stored on a server remote from the apparatus of FIG. 1 and the processing circuitry 32 sends data to and receives data from the model 42 via the API circuitry 34 which is configured to send suitable prompts or other instructions and/or data to the model 42 and to receive output from the model 42, under control of the processing circuitry 32. The API circuitry is able to communicate with the model 42 via a networked connection, for example via the internet, or via any other suitable direct or indirect connection. In alternative embodiments, the model 42 is stored locally at the apparatus of FIG. 1 rather than being stored remotely.

In various embodiments, the model 42 may comprise a transformer or other type of deep learning architecture that is configured to process text sequences. The model 42 may comprise a Generative Pre-trained Transformer (GPT) The model may be a chatbot such as the Chat Generative Pre-trained Transformer (ChatGPT). Any other suitable LLM may be used in other embodiments, for example at least one of GPT-2, GPT-3.5, GPT-4, PaLM, LLaMa, BLOOM, Ernie, T5, Claude or Claude 2, or any suitable derivatives or developments thereof.

The first input text 40 may be referred to as a first user prompt or user query. The first user prompt may condition the output of the model 42. The first user prompt may comprise text and be composed in a conversational format. The first user prompt may request the model 42 to perform one of more tasks relating to the processing of the first input text 40. The first input text 40 may, for example, comprise at least one of medical notes for a patient or other subject, results of a diagnostic or other procedure, test or scan results or text associated with such results.

The model 42 processes the first input text 40 to extract at least one item. In this embodiment the extracted items are referred to as headers, and the model 42 generate a list of one or more headers 44 and a list of one or more supporting quotes 46 from the first text 40. Collections of data other than lists may also be used. The supporting quotes 46 may comprise one or more subsets of the first input text 40 that is selected by the model 42. The supporting quotes 46 may be sentences of text from the first input text 40 selected by the model 42. The selection of the supporting quotes 46 may be based on the first user prompt or query that may form part of the first input text 40. The headers 44 may comprise a subset of the first input text 40 that is selected by the model 42. The selection of the headers may be based on the first user prompt that may form part of the first input text 40. The one or more headers 44 may further comprise a subset or shortening of the supporting quotes 46.

The selection of headers that comprise a subset of or a shortening of the supporting quotes may be performed by the model 42 on the basis of the first user prompt. The supporting quotes 46 and the headers 44 may be related based on a notion of similarity or relation between the two. The text of the supporting quotes 46 may support the text of the headers 44 in the context of the input prompt. The relationship that exists between the headers 44 that correspond to the supporting quotes 46, and is the criterion for their selection by the model 42 may be defined by the first user prompt or query.

The headers 44 are provided to the model 42 for processing in addition to a second input text 48. The second input text 48 may comprise structured or unstructured text or a combination of structured and unstructured text. The second input text 48 may comprise a second user prompt or query that conditions the output of the model 42. The model 42 processes the second input text 48 to generate a list of one or more second supporting quotes 52. The second supporting quotes 52 may comprise one or more subsets of the first second text 48 that is selected by the model 42. The second supporting quotes 52 may be sentences of text from the second input text 48 selected by the model 42. The selection of the second supporting quotes 52 may be based on the second user prompt or query that may form part of the second input text 40.

In the current embodiment, the second user prompt conditions the output of the model 42 to find a match between the headers 44 and the second input text 48. The model 42 processes the second input text 48 and headers 44 to obtain status of match 50 and select second supporting quotes 52 derived from the second input text 40 matched with each of the one or more headers 44. The list of status of match 50 comprises expressions, for example binary expressions, of whether there is a match between the headers 44 and the second input text 48 or there is no match between the two. The second supporting quotes 52 comprise one or more subsets of the second input text 48 that are selected by the model 42 and which correspond to the headers 44. The relationship between the headers 44 that correspond to the second supporting quotes 52 and is the criterion for their selection by the model 42 may be defined by the second user prompt or query.

The output data of method 200 is the combined textual data contained headers 44, supporting quotes 46, status of match 50 and second supporting quotes 52. The text data that is obtained from the output of method 200 consists of a subset of first text linked to a matching subset of a second text. The headers 44 and their associated second supporting quotes 52 are considered linked. In the present embodiment, this data/method may be able to automatically combine medical data from separate sources or separate sections of the same source that matches according to a user based criterion defined by user prompts. This automatic collation of medical data, as applied to medical reports of a user, may be beneficial in accelerating diagnoses by finding patterns that would otherwise be difficult to observe in large amounts of textual medical data.

The method 200 in this embodiment comprises two stages of providing input to a model 42 and two output stages. In other embodiments, the method may comprise further rounds of new input data and processing of the new data and previous data, such as three or four or more rounds.

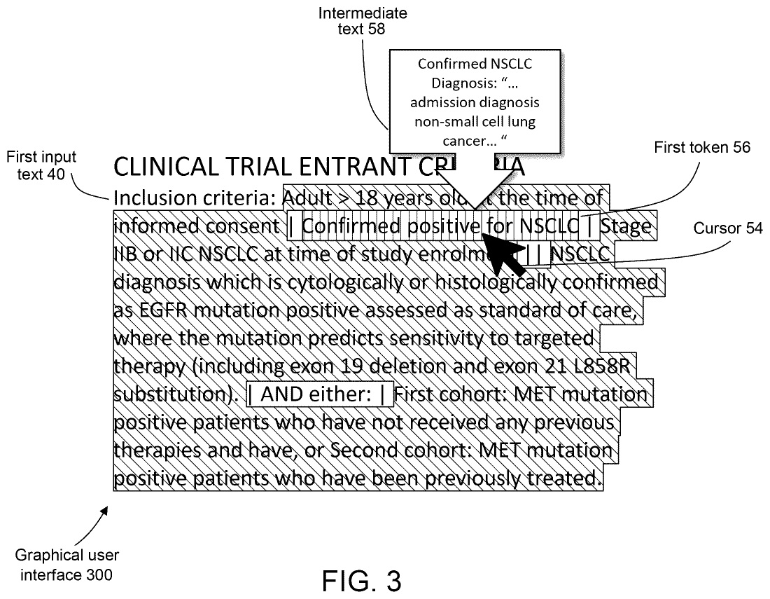

FIG. 3 is an image of a graphical user interface 300 in accordance with an embodiment. FIG. 3 is an image of a first input text 40 with visual features added and illustrates some of the output data of method 200 in the embodiment of FIG. 2. While the text illustrated in FIG. 3 comprises medical reports, any other modality of text may be used in other embodiments.

FIG. 3 shows a cursor 54 that is collocated with a first token 56 or quote of the first input text 40. In this particular embodiment, a token is represented by a sentence. In other embodiments, tokens may be represented by words, phrases, paragraphs or sections thereof. The sentence or token that is collocated with the cursor 54 is highlighted because the model 42 has determined that the matching condition, discussed later, is satisfied. A pop-up text box labelled ‘intermediate text’ 58 is generated showing the header 44 and the second supporting quote associated with it. The second input text 48 used to find matches with the headers 44 is not shown in FIG. 3. Considering the remainder of the text, second supporting quotes 52 which match the first token 56 or quote are highlighted. The tokens or quotes that do not match the first token 56 are highlighted differently. The highlighting in FIG. 2 is illustrated using different types of hatching or shading. In various embodiments, a tooltip, popover or mouse-over functionality may be used in the representing of, for example to cause display of, relevant parts of the first or second medical text. In alternative embodiments, any other suitable method of drawing attention to text or parts of text, for example quotes, may be used as well as instead of color highlighting, for example use of indicators such as shading, increasing or decreasing size of text, use of pointers or other graphical indicators or any other suitable indicators.

Any desired matching process may be performed by the processing circuitry, or the trained model under instruction from the processing circuitry, for example determining whether or not there is a match may comprise determining whether cognitive or semantic content of the extracted at least one item is the same as or consistent with at least part of the content of the second medical text. Alternatively or additionally, determining whether or not there is a match may comprise at least one of determining at least one criterion from the extracted at least one item and determining whether content of the second medical text complies with the at least one criterion; or determining whether or not there is a match between the extracted at least one item and content of the second medical text comprises determining a question represented by or comprised in the at least one item and determining whether the response to the question is positive or negative based on the second medical text.

As shown in FIG. 3, the original text may be linked to a second text and directly overlaid with related quotes, and visually marked as to the status of the match using a graphical user interface implementation.

One feature of method 200 is that it allows any linkages with no associated quote (or a quote which is hallucinated by the LLM) to be hidden from the user, and a transparent presentation of what spans in the input have been identified.

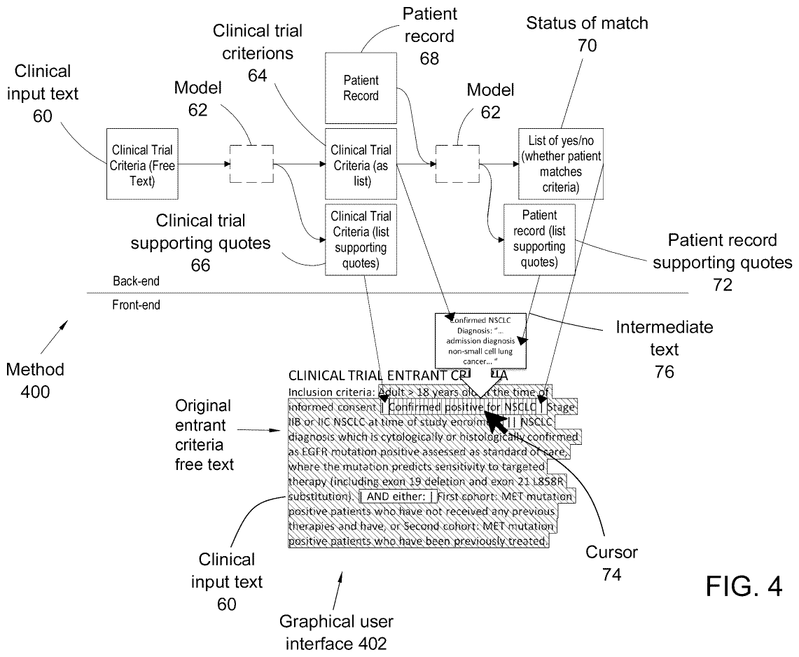

FIG. 4 is a schematic of a method 400 of processing text data and an image of the associated graphical user interface in accordance with an embodiment.

FIG. 4 comprises a schematic of a method 400 of processing medical text data in accordance with an embodiment. With reference to FIG. 4, a clinical input text 60 is provided to the model 42. The clinical input text may be structured text or unstructured free text. The clinical input text 60 may comprise clinical trial criteria. The clinical input text 60 may comprise medical data such as medical reports and patient records relevant to one or more users.

The first input text 60 may also contain a first user prompt. The first user prompt may condition the output of the model 42. The first user prompt may comprise text and be composed in a conversational format. The first user prompt may request the model 42 to perform one of more tasks relating to the processing of the clinical input text 60.

The model 42 processes the clinical input text 60 to generate a list of one or more clinical trial criteria 64 and a list of one or more clinical trial supporting quotes 66 for the clinical trial criteria. Collections of data other than lists may also be used. The clinical trial supporting quotes 66 may comprise one or more subsets of the first input text 60 that is selected by the model 42. The selection of the one or more supporting quotes 66 may be based on the first user prompt that may form part of the clinical input text 60. The clinical trial criteria 64 may comprise a subset of the first input text 60 that is selected by the model 42.

The selection of the clinical trial criteria 64 may be based on the first user prompt or query that may form part of the clinical input text 60. The one or more clinical trial criteria 64 may further comprise a subset or shortening of the clinical trial supporting quotes 66. The selection of headers that comprise a subset of, or a shortening of the supporting quotes may be performed by the model 42 on the basis of the first user prompt. The clinical trial supporting quotes 66 and the clinical trial criteria 64 may be related based on a notion of similarity or relation between the two. The text of the clinical trial supporting quotes 66 may support the text of the clinical trial criteria 64 in the context of a ground truth represented by them. The relationship between the clinical trial criteria 64 that correspond to the clinical trial supporting quotes 66 and is the criterion for their selection by the model 42, may be defined by the first user prompt or query.

The clinical trial criteria 64 are provided to the model 42 for processing in addition to a patient record 68. The patient record 68 may comprise structured or unstructured free text. The patient record 68 may comprise a second user prompt that conditions the output of the model 42.

In the current embodiment, the second user prompt or query conditions the output of the model 42 to find a or match between the clinical trial criteria 64 and the patient record 68. The model 42 processes the medical record 68 and clinical trial criteria 64 to obtain status of match 70 and ‘patient record supporting quotes’ 42 for each of the one or more clinical trial criteria 64 in the form of lists or other collections of textual data. The list of status of match 70 comprises binary expressions of whether there is a match between the clinical trial criteria 64 and patient record 68 or there is no match between the two. The second patient record supporting quotes 42 comprise one or more subsets of the patient record 68 that are selected by the model 42 and which correspond to the clinical trial criteria 64. The relationship between the clinical trial criteria 64 that correspond to the patient record supporting quotes 42 and is the criterion for their selection, by the model 42 may be defined by the second user prompt or query.

The output data of method 400 is the combined textual data contained in the clinical trial criteria 64, clinical trial supporting quotes 66, status of match 70 and patient record supporting quotes 72. The text data that is obtained from the output of method 400 consists of a subset of first text linked to a matching subset of a second text. This automatic collation of medical data, as applied to medical reports of a user, may be beneficial in accelerating diagnoses by finding patterns that would otherwise be difficult to observe in large amounts of textual medical data.

The method 400 in this embodiment comprises two stages of providing input to a model 42 and two output stages. In other embodiments, the method may comprise further rounds of new input data and processing of the new data and previous data.

FIG. 4 also shows a subsection of a graphical user interface 402 associated with the method 400. The graphical user interface 402 is shown as the clinical input text 60 overlaid with various visual markers that will be described below. In the present embodiment, the clinical input text 60 is shown separated into quotes. It will be assumed for the purpose of this discussion that the method 400 has been completed according to FIG. 4 and all data derived from the method is available. The act of collocating the cursor 74 with one particular supporting quote selects the supporting quote. The position of the cursor 74 also causes an intermediate text 76 to be overlaid on the clinical input text 60. The intermediate text 76 comprises the clinical trial criterion 64 associated with the supporting quote selected by the cursor 74. The clinical trial criterion 64 in this particular embodiment reads “Confirmed NSCLC diagnosis:” while the supporting quote reads “Confirmed positive for NSCLC”. It can be seen, as discussed earlier, that the clinical trial criterion is a subset or shortening of the text of the supporting quote. The intermediate text 76 also comprises a patient record supporting quote 72 that was selected from the patient record 68. As discussed earlier, the selection of the patient record supporting quote 72 is based on the clinical input text 60 and the patient record text 68, including the user prompts associated with both. The supporting quotes that are not associated with the clinical input text 60 and the patient record 68 are highlighted in the graphical user interface 300. The patient record supporting quote 72 in this embodiment reads “ASMISSION DIAGNOSIS: NON SMALL-CELL LUNG CANCER.”.

The text of the supporting quote ‘Confirmed positive for NSCLC’ has been summarised by the model 42 as ‘Confirmed NSCLC diagnosis’. Further processing the patient record 68 has resulted in the binary decision of match or linkage deemed as met by GPT resulting in the supporting quote being highlighted and accompanied by the patient record supporting quote 72 ‘ . . . admission diagnosis non-small cell lung cancer . . . ’. The model 42 has correctly linked the abbreviation NSCLC to non-small cell lung cancer.

This allows any linkages with no associated quote (or a quote which is hallucinated by the LLM) to be hidden from the user, and a transparent presentation of what spans in the input have been identified.

Moving the cursor 74 to the location of a different supporting quote in the clinical input text 60 will result in the selection of the supporting quote and the updating of the intermediate text 76. The new intermediate text 76 in this case will comprise a clinical trial criterion 64 and a patient record supporting quote 72 associated with the selected supporting quote. If the model 42 decides that there is a match between clinical trial criteria 64 and the patient record 68 associated with the selected supporting quote, the supporting quote will be highlighted while supporting quotes that do not fit the matching criterion will be highlighted differently. The highlighting in FIG. 4 is illustrated using different types of hatching or shading.

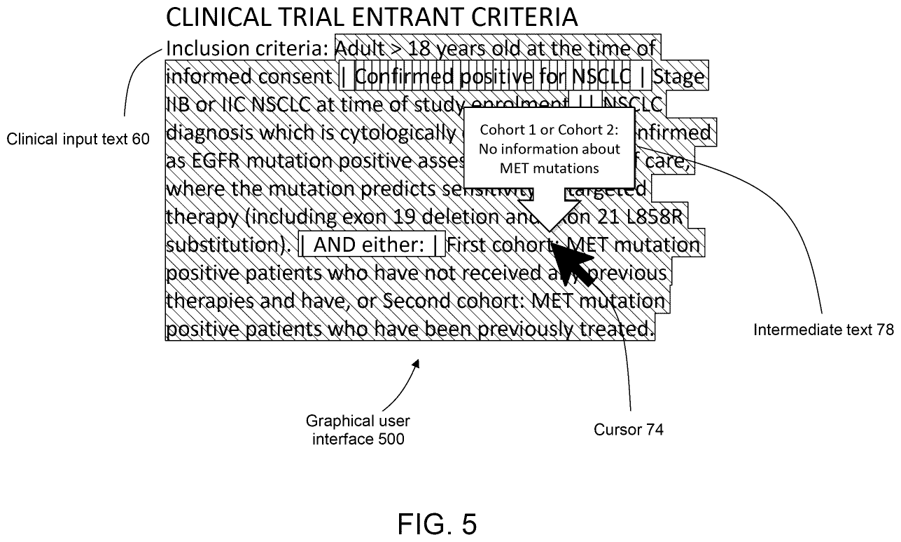

FIG. 5 shows an image of a graphical user interface 500 that can be used to parse the data generated by method 400 and method 200.

Here, specific details for cohort 1 and cohort 2 are listed, based on MET mutation status. Due to there being no information on MET status in the patient record, this criteria is marked using corresponding hatching or shading. In FIG. 5, the cursor is collocated with a supporting quote that reads “First cohort: MET mutation positive patients who have not received any previous therapies and have, or Second cohort: MET mutation positive patients who have been previously treated”. The intermediate text 78 comprises the associated clinical trial criterion 64 which reads “Cohort 1 or Cohort 2”. The intermediate text 78 also contains the status “No information about MET mutations”. It can be seen that because the model 42 was unable to find any text in the patient record 68 that matched the clinical trial criterion 64. For this reason, the supporting quote is highlighted differently. If the second text contains no information about MET mutations, a second quote is not presented in this example. Rather, ‘No information about MET mutation’ is presented and this is linked to the first supporting quote. The highlighting in FIG. 5 is illustrated using different types of hatching or shading

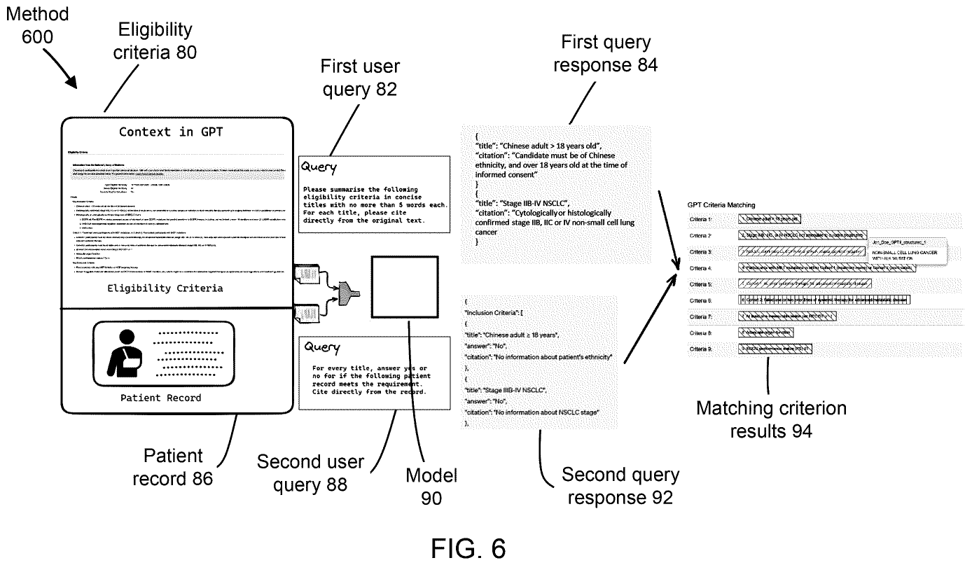

FIG. 6 illustrates a method 600 for processing text for using a model to match text from two sources. In other embodiments, more than two sources may be used. A first text labelled eligibility criteria 80 and a first user query 82 are provided to a model 90. In this embodiment, the model 90 is a Generative Pre-trained Transformer (GPT). In other embodiments, the model 90 may be any other LLM. The first user query 82 reads “Please summarise the following eligibility criteria in concise titles with no more than 5 words each. For each title, please cite directly from the original text.”. The model outputs a first query response 84 that comprises ‘titles’ and ‘citations’. Titles are similar to headers 44 of method 200 and clinical trial criteria 64 of method 400 while citations are similar to supporting quotes 46 of method 200 and clinical trial supporting quotes 66 of method 400. Patient record 86 and second user query 88 are also provided to the model 90 resulting in the output second query response 92. The second user query 88 reads “For every title, answer yes or no for in the following patient record meets the requirement. Cite directly from the record.”.

This causes the model 90 to generate titles and citations from the text of the patient record 86 accompanied by an answer as either a ‘yes or a ‘no’ in response to the second user query 88. The titles correspond to headers in FIG. 2. Titles or headers are also generated in methods 200 (FIG. 2) and 400 (FIG. 4) for the second input text. For example, the LLM may be asked, for instance in a clinical trial matching example, to summarise the criteria into headers/titles with supporting quotes for each.

The output of the method is the matching criterion results 94 which illustrates a graphical user interface combining the results of the model 90. The graphical user interface shows a list of criteria that may be color coded or shaded to represent the answer the matching query in the second user query 88. Colors or shadings may be selected and GPT may be asked to provide output in a structured format. The status of match (yes, no, unknown) can for example be mapped to a corresponding color or shading according to any desired color or shading scheme.

According to an embodiment there may be provided the following steps: Step 1 (clinical trial text): Ask the LLM to summarise the eligibility criteria into headers and quotes. Step 2 (patient text): match the headers to a patient record with evidence (quotes) from the patient record. From here, quotes from the clinical trial text are matched with relevant quotes from the patient record.

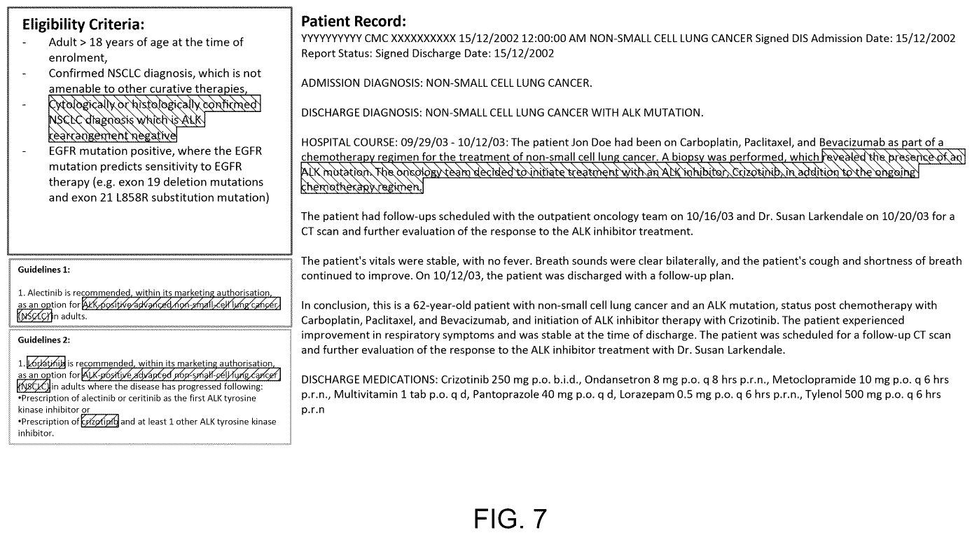

FIG. 7 shows an output provided to a user via a user interface according to a further embodiment. In this example, the first medical text comprises the combination of the Eligibilty Criteria document and the Guidelines 1 and Guidelines 2 documents shown in FIG. 7. The second medical text comprises the Patient Record document shown in FIG. 7. In this example, the items extracted from the first medical text and highlighted using highlighting in FIG. 7 are the text “Cytologically or histologically confirmed NSCLC diagnosis which is ALK rearrangement negative” from the Eligibility Criteria and the text “ALK-positive advanced non-small-cell lung cancer (NSCLC) from Guidelines 1, and the text “Lorlatinib”, “(ALK)-positive advanced non-small-cell lung cancer (NSCLC)” and “crizotinib” from Guidelines 2. A corresponding part of the second medical text that provides a match is highlighted using shading in FIG. 7 and comprises the text “revealed the presence of an ALK mutation. The oncology team decided to initiate treatment with an ALK inhibitor, Crizotinib, in addition to the ongoing chemotherapy regimen”. In this example, shading or hatching is used to represent a clinical trial match or a match with clinical guidelines, rather than the nature of the match (e.g. rather than yes, no, unknown as previously). In this example the eligibility criteria match has been given priority.

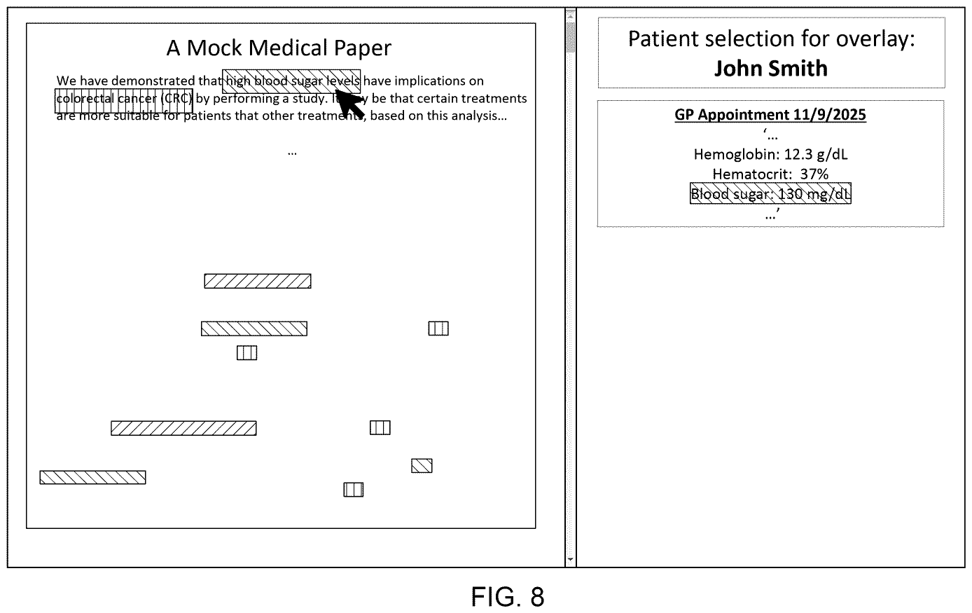

FIG. 8 shows an output provided to a user via a user interface according to another embodiment. The process is similar to that for FIG. 2, although for FIG. 2 the first text is a paper, and the text comprises GP records In the example of FIG. 8 case, the first medical text comprises a mock medical paper. The first medical text is shown as boxes in the figure (with some text blanked out) but it can be understood that in reality it includes text, including “high blood sugar levels” and “colorectal cancer (CRC)”. The second medical text comprises blood measurements obtained for patient John Smith at a GP appointment of 11 Sep. 2025. Various items have been extracted from the first medical text, each of which can be highlighted in the first medical text (highlighting is included in FIG. 8 but the underlying word(s) or passages of text being highlighted are not all shown to avoid reproducing the full text of the paper in the present document). The processing circuitry has determined that there is a match between the extracted item of a high blood sugar criterion from the first medical text and the blood sugar measurement result of 130 mg/dL of the second medical text. Quotes from the first medical text that correspond to the extracted item concerned (“high blood sugar levels”) are selected, and a quote is selected from the second medical text that provides a representation of the part of the second medical text that provides a match. The quotes corresponding to the extracted item from the first medical text and the part of the second medical text that matches are highlighted on the user interface of FIG. 8. Here, color, hatching or shading can be used to distinguish relevant diseases/symptoms. For example, when highlighting text in the documents, a first color, hatching or shading could be used to highlight CRC, a second color, hatching or shading could be used to highlight high glucose in the blood, and a third color, hatching or shading could be used to highlight diabetes. Rather than trying to present the nature of the match with color, hatching or shading (e.g. finding a CRC diagnosis in the patient records), in this example it is chosen to just show any match and to select color, hatching or shading based on the topic of the match.

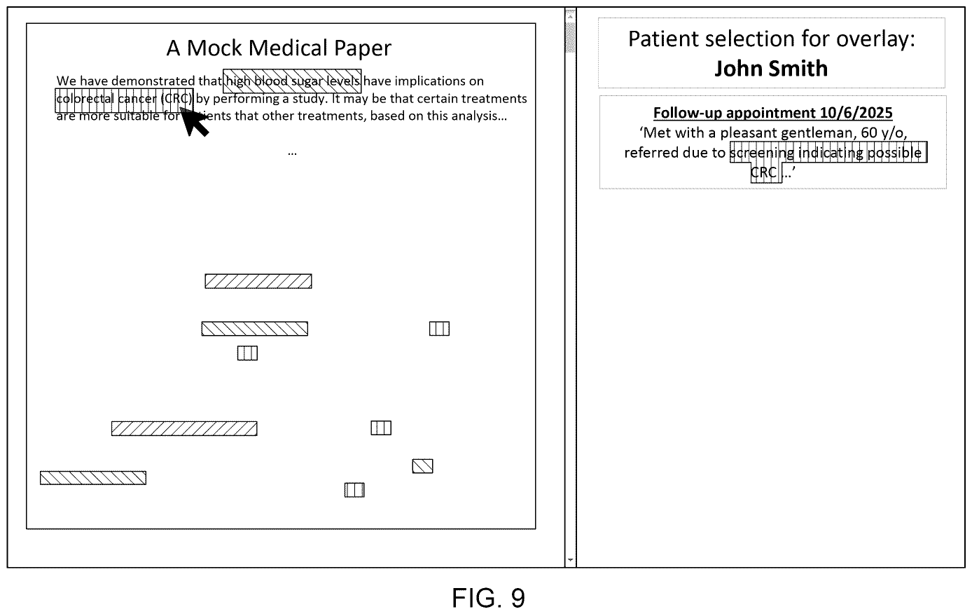

FIG. 9 shows an output provided to a user via a user interface according to another embodiment. FIG. 9 is similar to FIG. 8 but illustrates that the UI may show different information based on cursor location. Again, the first medical text comprises the paper from the mock medical paper. The second medical text comprises notes from a GP appointment of 10 Jun. 2025 for the same patient as for the embodiment of FIG. 8, namely John Smith. Various items have been extracted from the first medical text, each of which is highlighted. The processing circuitry has determined that there is a match between the extracted item of colorectal cancer from the first medical text and the part of the second medical text that mentions “screening indicating possible CRC”. Quotes from the first medical text that correspond to the extracted item concerned “colorectal cancer”, “CRC”) are selected, and a quote is selected from the second medical text (“screening indicating possible CRC”) that provides a representation of the part of the second medical text that provides a match. The quotes corresponding to the extracted item from the first medical text and the part of the second medical text that matches can be highlighted in color, hatching or shading on the user interface of FIG. 9.

According to various embodiments there is provided a system comprising processing circuitry configured to automatically match two medical texts (or two pluralities of medical texts) by:
summarising the source input medical texts into items, with supporting quotes for each item,
linking the plurality of items to items in a second target input medical text(s), with supporting quotes from the second text, and
presenting, through a user interface, the source text with the identified quotes overlaid with quotes from the target text.

The summarisation and quotes may be extracted by a Large Language Model (LLM). The LLM may be fine-tuned, and/or provided with example input output pairs. The status of the match between the source and target texts may be indicated visually by colorisation or shading of the text on the user interface. The graphical user interface may display the quotes from the target text on the source text by the use of tooltip, popover or mouse-over functionality. The input texts may be unstructured, structured or a mixture of structured and unstructured. The linkage/matching may be between a source clinical trial eligibility criteria and a target patient record, where a patient record may comprise a plurality of medical documents. The linkage/matching may be between source medical guidelines and a target patient record, where a patient record may comprise a plurality of medical documents. The linkage/matching may be between a source medical paper and a target patient record.

Various embodiments have been described in which supporting quotes linked to items are displayed via a user interface. In various embodiments the supporting quotes and highlighted items can be used in any other desired way, for example in planning of procedures such as a scanning plan or prescribing of drugs. The user interface may be included in, or accessible to, for example a scanner, or scan management software or a prescription management system in some embodiments, and the medical texts may include scan protocol texts, or scan instructions, or prescribing notes or workflows.

According to various embodiments there is provided medical data processing apparatus comprising processing circuitry configured to:
receive a first medical text and extract at least one item from the first medical text;
receive a second medical text and determine whether or not there is a match between the extracted at least one item and content of the second medical text including if there is a match determining a part of the second text that matches the extracted at least one item.

The system may further comprise a user interface configured to display at least part of the first medical text including displaying and/or highlighting the extracted at least one item.

The user interface may be configured also to display a representation of the part of the second medical text that matches the extracted at least one item, and to associate on the user interface the representation of the part of the second text and the matching extracted at least one item.

The associating on the user interface of the representation of the part of the second text and the matching extracted at least one item may comprise overlaying, linking or displaying in proximity the part of the second text and the matching extracted at least one item.

The representation of the part of the second medical text may comprise a quote from the second medical text.

The representation of the part of the second medical text may be displayed using a tooltip, popover or mouse-over functionality.

The user interface may be configured to output an indication whether there is a match or not between the extracted at least one item and content of the second medical text.

The indication may comprise at least one of highlighting text or display of different color(s), hatching, shading or indicator(s) depending on whether or not there is a match.

Determining whether or not there is a match between the extracted at least one item and content of the second medical text may comprise determining whether cognitive or semantic content of the extracted at least one item is the same as or consistent with at least part of the content of the second medical text.

Determining whether or not there is a match between the extracted at least one item and content of the second medical text may comprise at least one of:
a) determining at least one criterion from the extracted at least one item and determining whether content of the second medical text complies with the at least one criterion; or
b) determining whether or not there is a match between the extracted at least one item and content of the second medical text comprises determining a question represented by or comprised in the at least one item and determining whether the response to the question is positive or negative based on the second medical text.

The extracting of at least one item from the first medical text comprises at least one of:
a) selecting at least part of the first medical text;
b) summarising content of the first medical text and generating said at least one item to represent the summarised content.

The processing circuitry may be configured to use a trained model to perform at least one of:
the extracting of at least one item from the first medical text;
the determining of whether or not there is a match between the extracted at least one item and content of the second medical text.

The trained model may comprise a large language model (LLM) or other language model.

The model comprises at least one of GPT-2, GPT-3.5, GPT-4, PaLM, LLaMa, BLOOM, Ernie, T5, Claude or Claude 2, or any suitable derivatives or developments thereof.

One or both of the first medical text and the second medical text may be unstructured, structured or a mixture of structured and unstructured.

One of the first medical text and the second medical text may comprise clinical trial eligibility criteria or medical guidelines and the other of the first medical text and the second medical text may comprise a patient record, wherein the patient record may comprise a plurality of medical documents.

One of the first medical text and the second medical text may comprise a source medical paper and the other of the first medical text and the second medical text may comprise a patient record, wherein the patient record may comprise a plurality of medical documents.

The apparatus may further comprise:
a data store that stores at least one of the a first medical text or the second medical text;
a display device configured to provide a user interface that outputs to a user an indication of the outcome of the determining whether or not there is a match; and
communication circuitry operable to communicate with at least one of the data store and an external trained model that is operable based on instructions or other communication from the processing circuitry to perform at least one of the extracting of at least one item from the first medical text or the determining of whether or not there is a match between the extracted at least one item and content of the second medical text, and to receive from the trained model results of the at least one of extracting or determining.

Various embodiments may provide a method of matching medical data texts comprising:
receiving a first medical text and extract at least one item from the first medical text;
receiving a second medical text and determine whether or not there is a match between the extracted at least one item and content of the second medical text including if there is a match determining a part of the second text that matches the extracted at least one item.

Various embodiments may provide a non-transitory computer program product storing computer-readable instructions that are executable to:
receive a first medical text and extract at least one item from the first medical text;
receive a second medical text and determine whether or not there is a match between the extracted at least one item and content of the second medical text including if there is a match determining a part of the second text that matches the extracted at least one item.

There is also provided a user interface and query process which matches a patient record to specific clinical trial criteria in a transparent fashion.

Whilst particular circuitries have been described herein, in alternative embodiments functionality of one or more of these circuitries can be provided by a single processing resource or other component, or functionality provided by a single circuitry can be provided by two or more processing resources or other components in combination. Reference to a single circuitry encompasses multiple components providing the functionality of that circuitry, whether or not such components are remote from one another, and reference to multiple circuitries encompasses a single component providing the functionality of those circuitries.

Whilst certain embodiments are described, these embodiments have been presented by way of example only, and are not intended to limit the scope of the invention. Indeed, the novel methods and systems described herein may be embodied in a variety of other forms. Furthermore, various omissions, substitutions and changes in the form of the methods and systems described herein may be made without departing from the spirit of the invention. The accompanying claims and their equivalents are intended to cover such forms and modifications as would fall within the scope of the invention.

1. A medical data processing apparatus comprising processing circuitry configured to:
receive a first medical text and extract at least one item from the first medical text;
receive a second medical text and determine whether or not there is a match between the extracted at least one item and content of the second medical text including if there is a match determining a part of the second text that matches the extracted at least one item.

2. A medical data processing apparatus according to claim 1, wherein the processing circuitry is configured to use a trained model to perform at least one of:
the extracting of at least one item from the first medical text;
the determining of whether or not there is a match between the extracted at least one item and content of the second medical text.

3. A medical data processing apparatus according to claim 2, wherein the trained model comprises a large language model (LLM) or other language model.

4. A medical data processing apparatus according to claim 3, wherein the model comprises at least one of GPT-2, GPT-3.5, GPT-4, PaLM, LLaMa, BLOOM, Ernie, T5, Claude or Claude 2, or any suitable derivatives or developments thereof.

5. A medical data processing apparatus according to claim 1, wherein the system further comprises a user interface configured to display at least part of the first medical text including displaying and/or highlighting the extracted at least one item.

6. A medical data processing apparatus according to claim 5, wherein the user interface is configured also to display a representation of the part of the second medical text that matches the extracted at least one item, and to associate on the user interface the representation of the part of the second text and the matching extracted at least one item.

7. A medical data processing apparatus according to claim 6, wherein the associating on the user interface of the representation of the part of the second text and the matching extracted at least one item comprises overlaying, linking or displaying in proximity the part of the second text and the matching extracted at least one item.

8. A medical data processing apparatus according to claim 6, wherein the representation of the part of the second medical text comprises a quote from the second medical text.

9. A medical data processing apparatus according to claim 6, wherein the representation of the part of the second medical text is displayed using a tooltip, popover or mouse-over functionality.

10. A medical data processing apparatus according to claim 5, wherein the user interface is configured to output an indication whether there is a match or not between the extracted at least one item and content of the second medical text.

11. A medical data processing apparatus according to claim 10, wherein the indication comprises at least one of highlighting text or display of different color(s), hatching, shading or indicator(s) depending on whether or not there is a match.

12. A medical data processing apparatus according to claim 1, wherein determining whether or not there is a match between the extracted at least one item and content of the second medical text comprises determining whether cognitive or semantic content of the extracted at least one item is the same as or consistent with at least part of the content of the second medical text.

13. A medical data processing apparatus according to claim 1, wherein determining whether or not there is a match between the extracted at least one item and content of the second medical text comprises at least one of:
a) determining at least one criterion from the extracted at least one item and determining whether content of the second medical text complies with the at least one criterion; or
b) determining whether or not there is a match between the extracted at least one item and content of the second medical text comprises determining a question represented by or comprised in the at least one item and determining whether the response to the question is positive or negative based on the second medical text.

14. A medical data processing apparatus according to claim 1, wherein the extracting of at least one item from the first medical text comprises at least one of:
a) selecting at least part of the first medical text;
b) summarising content of the first medical text and generating said at least one item to represent the summarised content.

15. A medical data processing apparatus according to claim 1, wherein one or both of the first medical text and the second medical text is unstructured, structured or a mixture of structured and unstructured.

16. A medical data processing apparatus according to claim 1, wherein one of the first medical text and the second medical text comprises clinical trial eligibility criteria or medical guidelines and the other of the first medical text and the second medical text comprises a patient record, wherein the patient record may comprise a plurality of medical documents.

17. A medical data processing apparatus according to claim 1, wherein one of the first medical text and the second medical text comprises a source medical paper and the other of the first medical text and the second medical text comprises a patient record, wherein the patient record may comprise a plurality of medical documents.

18. An apparatus according to claim 1, further comprising:
a data store that stores at least one of the a first medical text or the second medical text;
a display device configured to provide a user interface that outputs to a user an indication of the outcome of the determining whether or not there is a match; and
communication circuitry operable to communicate with at least one of the data store and an external trained model that is operable based on instructions or other communication from the processing circuitry to perform at least one of the extracting of at least one item from the first medical text or the determining of whether or not there is a match between the extracted at least one item and content of the second medical text, and to receive from the trained model results of the at least one of extracting or determining.

19. A method of matching medical data texts comprising:
receiving a first medical text and extract at least one item from the first medical text;
receiving a second medical text and determine whether or not there is a match between the extracted at least one item and content of the second medical text including if there is a match determining a part of the second text that matches the extracted at least one item.

20. A non-transitory computer program product storing computer-readable instructions that are executable to:
receive a first medical text and extract at least one item from the first medical text;
receive a second medical text and determine whether or not there is a match between the extracted at least one item and content of the second medical text including if there is a match determining a part of the second text that matches the extracted at least one item.
